(19) United States
(12) Patent Application Publication
(10) Pub. No.: US 2018/0171403 A1
(43) Pub. Date: Jun. 21, 2018

(54) METHOD FOR DETERMINING THE RISK OF PRETERM LABOR

(71) Applicant: Meribank Biotech Co., Ltd, New Taipei City (TW)

(72) Inventors: Chang-Yo HSUAN, Taipei City (TW); Willie LIN, Taipei City (TW); Wei-Ting LIU, Taipei City (TW)

(73) Assignee: Meribank Biotech Co., Ltd, New Taipei City (TW)

(21) Appl. No.: 15/831,832

(22) Filed: Dec. 5, 2017

Related U.S. Application Data
(60) Provisional application No. 62/436,802, filed on Dec. 20, 2016.

(51) Int. Cl. C12Q 1/6876

(52) U.S. Cl. CPC C12Q 1/6876; C12Q 2600/118; C12Q 2600/158

(57) ABSTRACT

Provided is a method for determining the risk of preterm labor in a pregnant female subject. The method comprises detecting the expression level of one or more markers selected from the group consisting of TMEM200A, TMEM108 and GXYLT2, in a sample derived from the pregnant female subject.

### FIELD OF THE INVENTION

The present invention pertains to a method for determining the risk of preterm labor in a pregnant female subject.

### BACKGROUND OF THE INVENTION

Preterm birth (PTB), or birth before 37 weeks of gestation period, is the major cause of neonatal mortality and morbidity worldwide. Approximately 70% of the neonatal deaths are due to preterm delivery [1]. Often defined as a complex, multifactorial syndrome, PTB can be categorized into “spontaneous” or “medically indicated” types. Seventy-percent of PTB cases are idiopathic, of which 45% is resulting from the spontaneous onset of labor (sPTB) and 25% from preterm premature rupture of membranes (PPROM). The remaining 30% are medically indicated preterm deliveries that are often preceded by complications including gestational diabetes mellitus (GDM), preeclampsia (PE), intrauterine growth restriction (IUGR), and infections such as chorioamnionitis [2]. Several mechanisms underlying PTB have been proposed, including stress-induced maternal and fetal hypothalamic-pituitary-adrenal (HPA) axis, genital infections and inflammation of decidua-amniochorion, decidual hemorrhage, or premature hormone change such as increased circulating CRH and relaxin levels [3]. However, the pathogenesis of preterm birth has not been fully unveiled. The current diagnostic methods of PTB include cervical length measurement (<30 mm) and fibronectin test in the cervicovaginal fluid, but the sensitivity and specificity of this method are both low, calling for identification of better biomarker that can render information on PTB risk [4]. It has been reported that interleukin-8 (IL-8) and C-reactive protein (CRP), two recently identified preterm biomarkers [5, 6].

Glycosylation is one of the key components influencing several signaling pathways implicated in cell survival and growth. The Notch signaling pathway plays a pivotal role in numerous cell fate specifications during metazoan development. Both Notch and its ligands are repeatedly glycosylated by the addition of sugar moieties, such as O-fucose, O-glucose, or O-xylose, to bring about structural and functional changes [7]. Recently, the authors have shown [8] that two members of the human glycosyltransferase 8 family (GT8), GXYLT2 (glucoside-xylosyltransferase 2), is able to transfer the first α1,3-linked xylose to O-glucosylated mammalian Notch EGF repeats. The enzymes exhibit about 50% identity at amino acid sequence level with differences most apparent in the stem region, but no differences in their acceptor specificity could be observed so far. GXYLT2 is, however, not able to elongate substrates containing the disaccharide Xyl-Glc-[8], indicating the requirement for an additional xylosyltransferase. Here, the authors describe the identification of another human member of the GT8 family, only distantly related to the previously analyzed GT8 members, encoding a xyloside-xylosyltransferase (XXYLT), which further elongates the Xyl-Glc-O-Ser disaccharide on Notch EGF repeats [9].

TMEM200A protein is expected to localize in various compartments (membrane, integral to membrane). There are only two articles specifically referring to this gene in PubMed [10, 11]. No phenotype has yet been reported to our knowledge: this gene's in vivo function is yet unknown. However, TMEM108 protein is expected to localize in various compartments (extracellular space, membrane, nuclear membrane, integral to membrane). There are four articles specifically referring to this gene in PubMed [12, 13, 14, 15]. As the same, no phenotype has yet been reported to our knowledge: this gene's in vivo function is yet unknown.

### BRIEF SUMMARY OF THE INVENTION

The present invention provides a method for determining the risk of preterm labor in a pregnant female subject, comprising detecting the expression level of one or more markers selected from the group consisting of TMEM200A, TMEM108 and GXYLT2, in a sample derived from the pregnant female subject.

According to the present invention, the sample may be selected from the group consisting of blood, urine, amniotic fluid, and mesenchymal stem or stromal cells (MSCs) derived from a placenta-related tissue. In certain embodiments of the present invention, the placenta-related tissue is selected from the group consisting of amniotic membrane, chorionic disk, chorionic membrane, and umbilical cord.

Accordingly to the present invention, the expression level is a protein expression level or an mRNA expression level.

In certain embodiments of the present invention, the detecting step is performed through a polymerase chain reaction, a real-time polymerase chain reaction, or a quantitative polymerase chain reaction.

According to certain embodiments of the present invention, the method further comprises comparing the expression level of the marker with a normal expression level. In one preferred embodiment, the normal expression level is an average expression level of the marker in a plurality of corresponding samples derived from a plurality of pregnant female subjects who deliver at full term.

According to one embodiment of the present invention, a lower expression level of marker TMEM200A indicates the pregnant female subject is at risk of preterm delivery. For example, the lower expression level is an expression level at least 5%, 10%, 20% or 30% lower than the normal expression level.

According to one embodiment of the present invention, a lower expression level of marker TMEM108 indicates the pregnant female subject is at risk of preterm delivery. For example, the lower expression level is an expression level at least 5%, 10%, 20% or 30% lower than the normal expression level.

According to another embodiment of the present invention, a lower expression level of marker GXYLT2 indicates the pregnant female subject is at risk of preterm delivery. For example, the lower expression level is an expression level at least 5%, 10%, 20% or 30% lower than the normal expression level.

It is to be understood that both the foregoing general description and the following detailed description are exemplary and explanatory only and are not restrictive of the invention.

### BRIEF DESCRIPTION OF THE SEVERAL VIEWS OF THE DRAWINGS

The foregoing summary, as well as the following detailed description of the invention, will be better understood when read in conjunction with the appended drawings. For the purpose of illustrating the invention, there are shown in the drawings embodiments which are presently preferred.

In the drawings:

FIG. 1A shows quantitative real-time polymerase chain reaction of CRP and TMEM200A transcript. Transcript levels of CRP and TMEM200A in preterm birth were evaluated by fold enrichment compared to the expression of CRP and TMEM200A in full-term birth, i.e. by the comparison of the CRP and TMEM200A mRNA level in preterm birth versus the CRP and TMEM200A mRNA level in average value of full-term birth. The CRP was as a positive biomarker. Transcript levels of CRP and TMEM200A in preterm birth were demonstrated by the samples from CK001, TSG002, CK004, CK005, LCG006, TSG008, LCG009, TSG010, LCG011, LCG012, CK013, LCG014, TSG015, CK016, CK017 and LCG018. Transcript levels of CRP and TMEM200A in full-term birth were demonstrated by the samples from donor #006, #012, #017, #021, #023, #025, #026, #028, #061, #066, #067 and #075. The results showed that CRP was highly enriched in preterm birth compared with full-term birth vitro. But TMEM200A was lower expressed in preterm birth compared with full-term birth vitro.

FIG. 1B shows quantitative real-time polymerase chain reaction of CRP and TMEM108 transcript. Transcript levels of CRP and TMEM108 in preterm birth were evaluated by fold enrichment compared to the expression of CRP and TMEM108 in full-term birth, i.e. by the comparison of the CRP and TMEM108 mRNA level in preterm birth versus the CRP and TMEM108 mRNA level in average value of full-term birth. The CRP was as a positive biomarker. Transcript levels of CRP and TMEM108 in preterm birth were demonstrated by the samples from CK001, TSG002, CK004, CK005, LCG006, TSG008, LCG009, TSG010, LCG011, LCG012, CK013, LCG014, TSG015, CK016, CK017 and LCG018. Transcript levels of CRP and TMEM108 in full-term birth were demonstrated by the samples from donor #006, #012, #017, #021, #023, #025, #026, #028, #061, #066, #067 and #075. The results showed that CRP was highly enriched in preterm birth compared with full-term birth vitro. But TMEM108 was lower expressed in preterm birth compared with full-term birth vitro.

FIG. 1C shows quantitative real-time polymerase chain reaction of CRP and GXYLT2 transcript. Transcript levels of CRP and GXYLT2 in preterm birth were evaluated by fold enrichment compared to the expression of CRP and GXYLT2 in full-term birth, i.e. by the comparison of the CRP and GXYLT2 mRNA level in preterm birth versus the CRP and GXYLT2 mRNA level in average value of full-term birth. The CRP was as a positive biomarker. Transcript levels of CRP and GXYLT2 in preterm birth were demonstrated by the samples from CK001, TSG002, CK004, CK005, LCG006, TSG008, LCG009, TSG010, LCG011, LCG012, CK013, LCG014, TSG015, CK016, CK017 and LCG018. Transcript levels of CRP and GXYLT2 in full-term birth were demonstrated by the samples from donor #006, #012, #017, #021, #023, #025, #026, #028, #061, #066, #067 and #075. The results showed that CRP was highly enriched in preterm birth compared with full-term birth vitro. But GXYLT2 was lower expressed in preterm birth compared with full-term birth vitro.

FIG. 2A shows the percentage decrease of gene expression (fold change) of TMEM200A. The TMEM200A expression level in preterm birth or full-term birth was divided by average TMEM200A expression level in fill-term birth. Then the fold change of expression levels were calculated its percentage decrease. By comparing to the percentage decrease in all population, the statistical analysis could be used to PTB diagnosis.

FIG. 2B shows the percentage decrease of gene expression (fold change) of TMEM108. The TMEM108 expression level in preterm birth or full-term birth was divided by average TMEM108 expression level in fill-term birth. Then the fold change of expression levels were calculated its percentage decrease. By comparing to the percentage decrease in all population, the statistical analysis could be used to PTB diagnosis.

FIG. 2C shows the percentage decrease of gene expression (fold change) of GXYLT2. The GXYLT2 expression level in preterm birth or full-term birth was divided by average GXYLT2 expression level in fill-term birth. Then the fold change of expression levels were calculated its percentage decrease. By comparing to the percentage decrease in all population, the statistical analysis could be used to PTB diagnosis.

### DETAILED DESCRIPTION OF THE INVENTION

The present invention provides a method for determining the risk of preterm labor in a pregnant female subject. The method comprises detecting the expression level of one or more markers selected from the group consisting of TMEM200A, TMEM108 and GXYLT2, in a sample derived from the pregnant female subject.

The sample includes but is not limited to blood, urine, amniotic fluid, and mesenchymal stem or stromal cells (MSCs) derived from a placenta-related tissue.

In certain embodiments of the present invention, the placenta-related tissue is selected from the group consisting of amniotic membrane, chorionic disk, chorionic membrane, and umbilical cord.

Accordingly to the present invention, the expression level is a protein expression level or an mRNA expression level. In certain embodiments of the present invention, the detecting step is performed through a polymerase chain reaction, a real-time polymerase chain reaction, or a quantitative polymerase chain reaction.

According to certain embodiments of the present invention, the method further comprises comparing the expression level of a marker of TMEM200A, TMEM108 or GXYLT2, with a normal expression level of said maker in pregnant female subjects who deliver at full term. Preferably, the normal expression level is an average expression level of the marker in a plurality of corresponding samples derived from a plurality of pregnant female subjects who deliver at full term.

As used herein, the term “expression level” refers to a protein expression level or an mRNA expression level.

According to one embodiment of the present invention, a lower expression level of marker TMEM200A indicates the pregnant female subject is at risk of preterm delivery. For example, the lower expression level is an expression level at least 5%, 10%, 15%, 20%, 25%, 30%, 35%, 40%, 45%, 50%, 55%, 60%, 65%, 70%, 75%, 80%, 85%, 90%, or 95% lower than the normal expression level.

According to one embodiment of the present invention, a lower expression level of marker TMEM108 indicates the pregnant female subject is at risk of preterm delivery. For example, the lower expression level is an expression level at least 5%, 10%, 15%, 20%, 25%, 30%, 35%, 40%, 45%, 50%, 55%, 60%, 65%, 70%, 75%, 80%, 85%, 90%, or 95% lower than the normal expression level.

According to another embodiment of the present invention, a lower expression level of marker GXYLT2 indicates the pregnant female subject is at risk of preterm delivery. For example, the lower expression level is an expression level at least 5%, 10%, 15%, 20%, 25%, 30%, 35%, 40%, 45%, 50%, 55%, 60%, 65%, 70%, 75%, 80%, 85%, 90%, or 95% lower than the normal expression level.

The present invention also contemplates determining the risk of preterm labor by the combination of two or all of the above-mentioned marks. For example, if two or all of the markers exhibit lower expression levels compared to the corresponding normal expression levels, it suggests that the pregnant female subject is at high risk of preterm delivery.

In certain embodiments of the present invention, the detecting step is performed through a polymerase chain reaction, a real-time polymerase chain reaction, or a quantitative polymerase chain reaction. For example, the detection may be done by a real-time polymerase chain reaction instrument (LightCycler® 480 II, Roche).

According to one embodiment of the present invention, the method further comprises the preliminary steps of collecting the MSCs from the placenta-related tissue, and culturing the MSCs in a culture medium to prepare the primary culture.

The present invention is further illustrated by the following examples, which are provided for the purpose of demonstration rather than limitation.

### EXAMPLES

### Example 1

### Quantitative Real-Time Polymerase Chain Reaction Evaluation of CRP, TMEM200A, TMEM108 and GXYLT2 Transcript in Placenta-Derived Mesenchymal Stem Cells (MSCs)

Total RNA from 28 populations of placenta-derived cells from female subjects (samples associated with preterm birth, n=16 and samples associated with full-term birth, n=12) were isolated using the Direct-zol miniprep Kit (Zymo Research Corporation, CA, USA). The complementary DNA (cDNA) was synthesized with Transcriptor First Strand cDNA Synthesis Kit (Roche, Basel, Switzerland). Then Quantitative RT-PCR was performed using the Roche SYBR Green System with a LightCycler480 II (Roche, Basel, Switzerland) according to the manufacturer's instructions.

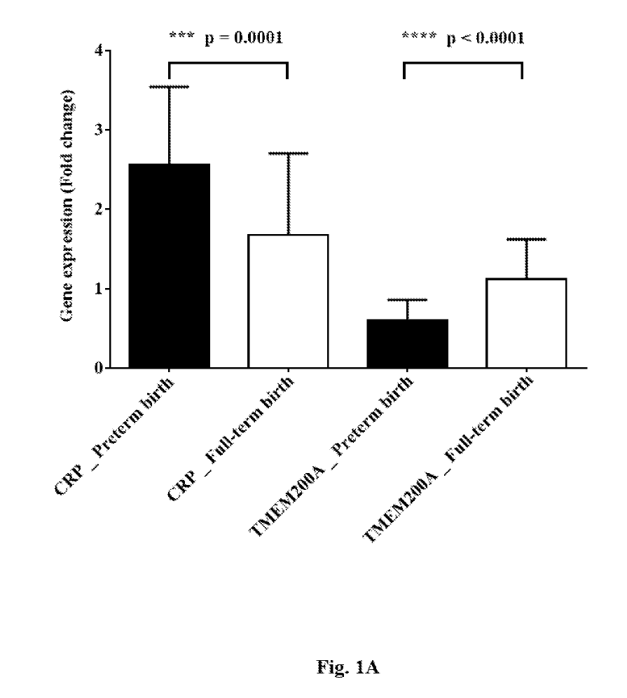

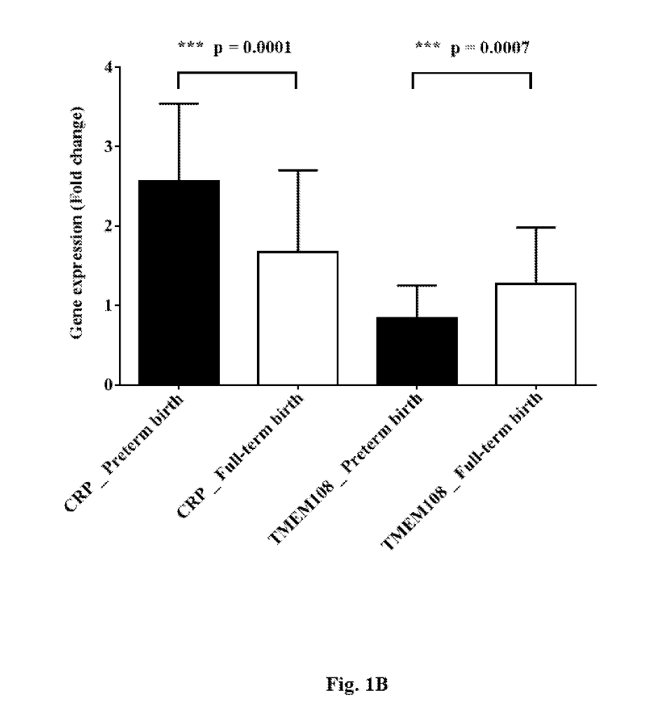

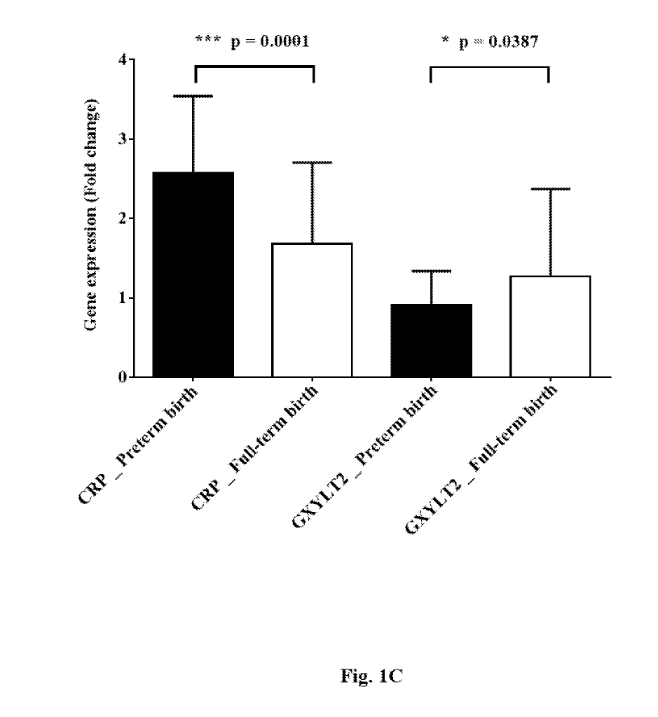

We assessed the expression of CRP (positive control) and TMEM200A by quantitative real-time polymerase chain reaction. Gene expression was normalized to the endogenous gene glyceraldehyde-3-phosphate dehydrogenase expression in the different cell populations. The expression of CRP and TMEM200A transcript in MSCs was calculated by fold enrichment compared to the average value of full-term birth. The results showed that CRP was expressed at a higher level in samples associated with preterm birth when compared with samples associated with full-term birth (FIG. 1A). On the other hand, the expression level of TMEM200A was significantly lower in the samples associated with preterm birth.

We also assessed the expression of CRP, TMEM108 and GXYLT2 by quantitative real-time polymerase chain reaction. Gene expression was normalized to the endogenous gene glyceraldehyde-3-phosphate dehydrogenase expression in the different cell populations. The expression of CRP, TMEM108 and GXYLT2 transcript in MSCs was calculated by fold enrichment compared to the average value of full-term birth. The results showed that CRP was expressed at a higher level in samples associated with preterm birth when compared with samples associated with full-term birth. But TMEM108 and GXYLT2 were expressed at a significantly lower level in samples associated with preterm birth when compared with samples associated with full-term birth (FIG. 1B and FIG. 1C).

### Example 2

### The Statistical Analyses for PTB Diagnosis

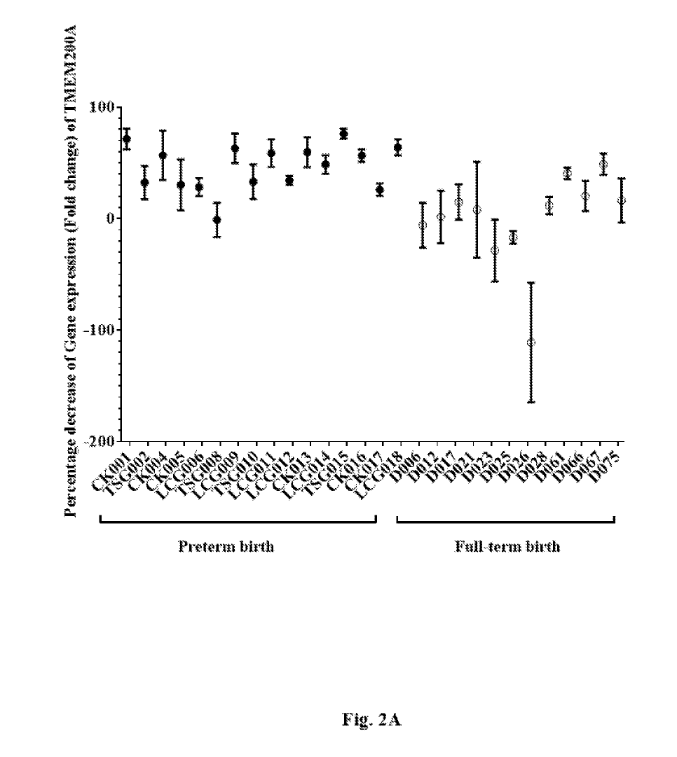

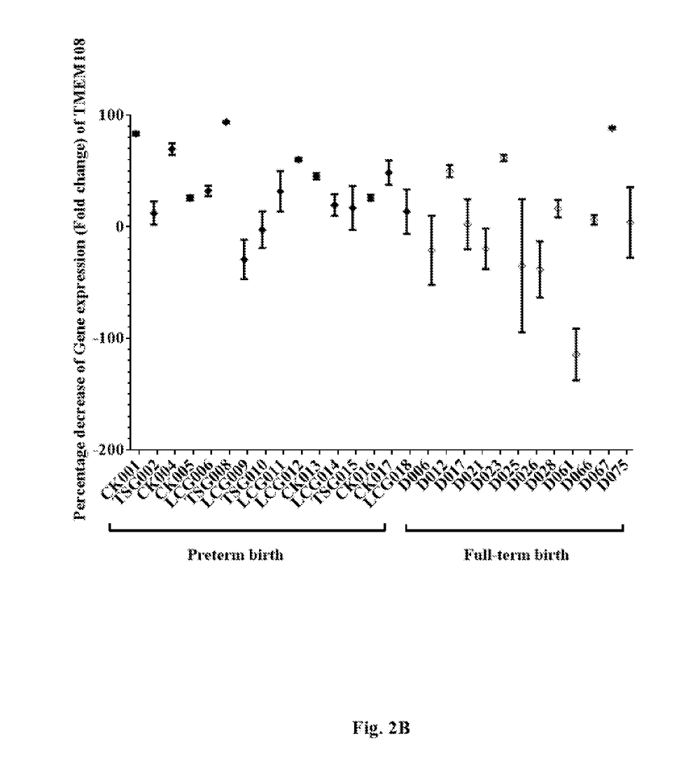

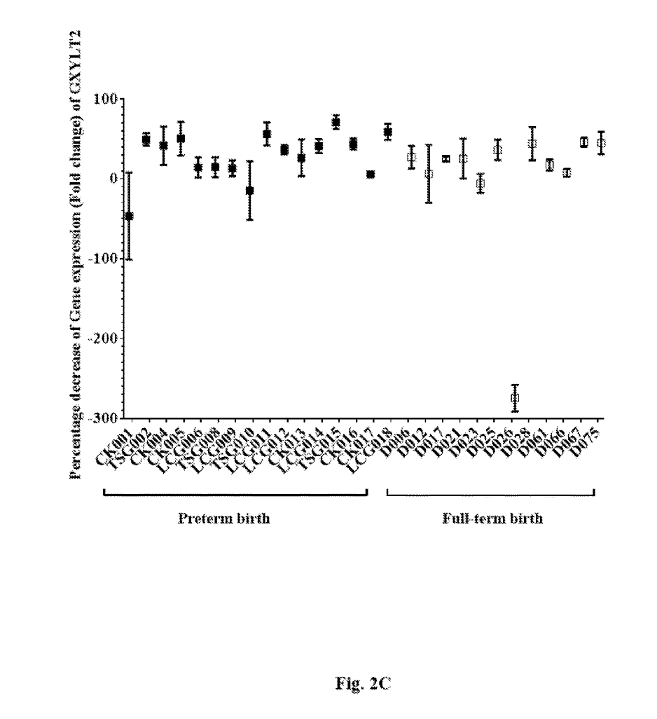

In order to evaluate the occurrence of PTB in all population, expression levels of TMEM200A, TMEM108 and GXYLT2 were determined by real-time polymerase chain reaction. Gene expression was normalized to the endogenous gene glyceraldehyde-3-phosphate dehydrogenase expression in the different cell populations. The expression of TMEM200A, TMEM108 and GXYLT2 transcript in MSCs was calculated by fold enrichment compared to the average value of full-term birth. The TMEM200A, TMEM108 and GXYLT2 expression levels in samples associated with preterm birth or full-term birth were divided by the average TMEM200A, TMEM108 and GXYLT2 expression levels, respectively, in samples associated with full-term birth. Then the fold change of expression levels were calculated as its percentage decrease (FIGS. 2A-2C).

Further, we assessed the percentage decrease from 10% to 90% in TMEM200A, TMEM108 and GXYLT2. The results are shown in Table 1 below.

TABLE 1
The diagnosis and misdiagnosis rate
of PTB detection in all population.

| | PTB detection | | | |
|---|---|---|---|---|
| | Preterm | Full-term | | |
| Percentage | birth | birth | Diagnosis | Misdiagnosis |
| Decrease | N = 16 | N = 12 | rate | rate |
| ≥10% decrease of | 15 (16) | 6 (12) | 94% | 50% |
| TMEM200A | | | | |
| ≥20% decrease of | 15 (16) | 3 (12) | 94% | 25% |
| TMEM200A | | | | |
| ≥30% decrease of | 13 (16) | 2 (12) | 81% | 17% |
| TMEM200A | | | | |
| ≥40% decrease of | 9 (16) | 2 (12) | 56% | 17% |
| TMEM200A | | | | |
| ≥50% decrease of | 8 (16) | 0 (12) | 50% | 0% |
| TMEM200A | | | | |
| ≥60% decrease of | 4 (16) | 0 (12) | 25% | 0% |
| TMEM200A | | | | |
| ≥70% decrease of | 2 (16) | 0 (12) | 13% | 0% |
| TMEM200A | | | | |
| ≥80% decrease of | 0 (16) | 0 (12) | 0% | 0% |
| TMEM200A | | | | |
| ≥90% decrease of | 0 (16) | 0 (12) | 0% | 0% |
| TMEM200A | | | | |
| ≥10% decrease of | 14 (16) | 4 (12) | 88% | 33% |
| TMEM108 | | | | |
| ≥20% decrease of | 10 (16) | 3 (12) | 63% | 25% |
| TMEM108 | | | | |
| ≥30% decrease of | 8 (16) | 3 (12) | 50% | 25% |
| TMEM108 | | | | |
| ≥40% decrease of | 6 (16) | 3 (12) | 38% | 25% |
| TMEM108 | | | | |
| ≥50% decrease of | 4 (16) | 3 (12) | 25% | 25% |
| TMEM108 | | | | |
| ≥60% decrease of | 4 (16) | 2 (12) | 25% | 17% |
| TMEM108 | | | | |
| ≥70% decrease of | 2 (16) | 1 (12) | 13% | 8% |
| TMEM108 | | | | |
| ≥80% decrease of | 2 (16) | 1 (12) | 13% | 8% |
| TMEM108 | | | | |
| ≥90% decrease of | 1 (16) | 0 (12) | 6% | 0% |
| TMEM108 | | | | |
| ≥10% decrease of | 13 (16) | 8 (12) | 81% | 67% |
| GXYLT2 | | | | |
| ≥20% decrease of | 10 (16) | 7 (12) | 63% | 58% |
| GXYLT2 | | | | |
| ≥30% decrease of | 9 (16) | 4 (12) | 56% | 33% |
| GXYLT2 | | | | |
| ≥40% decrease of | 8 (16) | 3 (12) | 50% | 25% |
| GXYLT2 | | | | |
| ≥50% decrease of | 4 (16) | 0 (12) | 25% | 0% |
| GXYLT2 | | | | |
| ≥60% decrease of | 1 (16) | 0 (12) | 6% | 0% |
| GXYLT2 | | | | |
| ≥70% decrease of | 1 (16) | 0 (12) | 6% | 0% |
| GXYLT2 | | | | |
| ≥80% decrease of | 0 (16) | 0 (12) | 0% | 0% |
| GXYLT2 | | | | |
| ≥90% decrease of | 0 (16) | 0 (12) | 0% | 0% |
| GXYLT2 | | | | |

We also assessed the combination of any two markers of TMEM200A, TMEM108 and GXYLT2 from above results. The results are shown in Table 2 below.

TABLE 2
The diagnosis and misdiagnosis rate of PTB detection
in any two genes of TMEM200A, TMEM108 and GXYLT2.

| | PTB detection | | | |
|---|---|---|---|---|
| | Preterm | Full-term | | |
| Percentage | birth | birth | Diagnosis | Misdiagnosis |
| Decrease | N = 16 | N = 12 | rate | rate |
| ≥20% decrease of | 13 (16) | 1 (12) | 81% | 8% |
| TMEM200A | | | | |
| ≥10% decrease of | | | | |
| TMEM108 | | | | |
| ≥20% decrease of | 9 (16) | 1 (12) | 56% | 8% |
| TMEM200A | | | | |
| ≥20% decrease of | | | | |
| TMEM108 | | | | |
| ≥20% decrease of | 12 (16) | 2 (12) | 75% | 17% |
| TMEM200A | | | | |
| ≥10% decrease of | | | | |
| GXYLT2 | | | | |
| ≥20% decrease of | 10 (16) | 1 (12) | 63% | 8% |
| TMEM200A | | | | |
| ≥20% decrease of | | | | |
| GXYLT2 | | | | |
| ≥30% decrease of | 11 (16) | 1 (12) | 69% | 8% |
| TMEM200A | | | | |
| ≥10% decrease of | | | | |
| TMEM108 | | | | |
| ≥30% decrease of | 7 (16) | 1 (12) | 44% | 8% |
| TMEM200A | | | | |
| ≥20% decrease of | | | | |
| TMEM108 | | | | |
| ≥30% decrease of | 11 (16) | 2 (12) | 69% | 17% |
| TMEM200A | | | | |
| ≥10% decrease of | | | | |
| GXYLT2 | | | | |
| ≥30% decrease of | 9 (16) | 1 (12) | 56% | 8% |
| TMEM200A | | | | |
| ≥20% decrease of | | | | |
| GXYLT2 | | | | |
| ≥10% decrease of | 12 (16) | 2 (12) | 75% | 17% |
| TMEM108 | | | | |
| ≥10% decrease of | | | | |
| GXYLT2 | | | | |
| ≥10% decrease of | 10 (16) | 2 (12) | 63% | 17% |
| TMEM108 | | | | |
| ≥20% decrease of | | | | |
| GXYLT2 | | | | |
| ≥20% decrease of | 8 (16) | 1 (12) | 50% | 8% |
| TMEM108 | | | | |
| ≥10% decrease of | | | | |
| GXYLT2 | | | | |
| ≥20% decrease of | 6 (16) | 1 (12) | 38% | 8% |
| TMEM108 | | | | |
| ≥20% decrease of | | | | |
| GXYLT2 | | | | |

Finally, the results of the combination of the three markers are shown in Table 3 below.

TABLE 3
The diagnosis and misdiagnosis rate of PTB detection
in the combination of TMEM200A, TMEM108 and GXYLT2.

| | PTB detection | | | |
|---|---|---|---|---|
| | Preterm | Full-term | | |
| Percentage | birth | birth | Diagnosis | Misdiagnosis |
| Decrease | N = 16 | N = 12 | rate | rate |
| ≥20% decrease of | 11 (16) | 1 (12) | 69% | 8% |
| TMEM200A | | | | |
| ≥10% decrease of | | | | |
| TMEM108 | | | | |
| ≥10% decrease of | | | | |
| GXYLT2 | | | | |
| ≥20% decrease of | 10 (16) | 1 (12) | 63% | 8% |
| TMEM200A | | | | |
| ≥10% decrease of | | | | |
| TMEM108 | | | | |
| ≥20% decrease of | | | | |
| GXYLT2 | | | | |
| ≥20% decrease of | 7 (16) | 1 (12) | 44% | 8% |
| TMEM200A | | | | |
| ≥20% decrease of | | | | |
| TMEM108 | | | | |
| ≥10% decrease of | | | | |
| GXYLT2 | | | | |
| ≥20% decrease of | 6 (16) | 1 (12) | 38% | 8% |
| TMEM200A | | | | |
| ≥20% decrease of | | | | |
| TMEM108 | | | | |
| ≥20% decrease of | | | | |
| GXYLT2 | | | | |
| ≥30% decrease of | 11 (16) | 1 (12) | 69% | 8% |
| TMEM200A | | | | |
| ≥10% decrease of | | | | |
| TMEM108 | | | | |
| ≥10% decrease of | | | | |
| GXYLT2 | | | | |
| ≥30% decrease of | 10 (16) | 1 (12) | 63% | 8% |
| TMEM200A | | | | |
| ≥10% decrease of | | | | |
| TMEM108 | | | | |
| ≥20% decrease of | | | | |
| GXYLT2 | | | | |
| ≥30% decrease of | 6 (16) | 1 (12) | 38% | 8% |
| TMEM200A | | | | |
| ≥20% decrease of | | | | |
| TMEM108 | | | | |
| ≥10% decrease of | | | | |
| GXYLT2 | | | | |
| ≥30% decrease of | 6 (16) | 1 (12) | 38% | 8% |
| TMEM200A | | | | |
| ≥20% decrease of | | | | |
| TMEM108 | | | | |
| ≥20% decrease of | | | | |
| GXYLT2 | | | | |

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and score of the present invention as defined by the appended claims.

### REFERENCES

[1] Shah J, Baxi B. Identification of biomarkers for prediction of preterm delivery. Journal of Medical society 2016:3-14.
[2] Eidem H R, Ackerman W Et, McGary K L, Abbot P, Rokas A. Gestational tissue transcriptomics in term and preterm human pregnancies: a systematic review and meta-analysis. BMC medical genomics 2015; 8:27.
[3] Kota S K, Gayatri K, Jammula S, Kota S K, Krishna S V, Meher L K, et al. Endocrinology of parturition. Indian journal of endocrinology and metabolism 2013; 17:50-9.
[4] Auray-Blais C, Raiche E, Gagnon R, Berthiaume M, J C. P. Metabolomics and preterm birth: What biomarkers in cervicovaginal secretions are predictive of high-risk pregnant women? Int J Mass Spectrom 2011; 307:33-8.
[5] Paananen R, Husa A K, Vuolteenaho R, Herva R, Kaukola T, Hallman M. Blood cytokines during the perinatal period in very preterm infants: relationship of inflammatory response and bronchopulmonary dysplasia. The Journal of pediatrics 2009; 154:39-43 e3.
[6] Ambalavanan N, Carlo W A, D'Angio C T, McDonald S A, Das A, Schendel D, et al. Cytokines associated with bronchopulmonary dysplasia or death in extremely low birth weight infants. Pediatrics 2009; 123:1132-41.
[7] Pakkiriswami S, Couto A, Nagarajan U, Georgiou M. Glycosylated Notch and Cancer. Front Oncol. 2016; 6:37.
[8] Sethi M K, Buettner F F, Krylov V B, Takeuchi H, Nifantiev N E, Haltiwanger R S, Gerardy-Schahn R, Bakker H. Identification of glycosyltransferase 8 family members as xylosyltransferases acting on O-glucosylated Notch epidermal growth factor repeats. J Biol Chem. 2010; 285, 1582-1586.
[9] Sethi M K, Buettner F F R, Ashikov A, Krylov V B, Takeuchi H, Nifantiev N E, Haltiwanger R S, Gerardy-Schahn R, Bakker H. Molecular Cloning of a Xylosyltransferase That Transfers the Second Xylose to O-Glucosylated Epidermal Growth Factor Repeats of Notch. J Biol Chem. 2012; 287 (4):2739-2748.
[10] Nagase T, Kikuno R, Ohara O. Prediction of the coding sequences of unidentified human genes. XXI. The complete sequences of 60 new cDNA clones from brain which code for large proteins. DNA Res. 2001; 8 (4):179-87.
[11] Pickard B S, Malloy M P, Clark L, Lehellard S, Ewald H L, Mors O, Porteous D J, Blackwood D H, Muir W J. Candidate psychiatric illness genes identified in patients with pericentric inversions of chromosome 18. Psychiatr Genet. 2005; 15 (1):37-44.
[12] Nagase T, Kikuno R, Hattori A, Kondo Y, Okumura K, Ohara O. Prediction of the coding sequences of unidentified human genes. XIX. The complete sequences of 100 new cDNA clones from brain which code for large proteins in vitro. DNA Res. 2000; 7 (6):347-55.
[13] Wiemann S, Weil B, Wellenreuther R, Gassenhuber J, Glassl S, Ansorge W, Böcher M, Blöcker H, Bauersachs S, Blum H, Lauber J, Düsterhöft A, Beyer A, Köhrer K, Strack N, Mewes H W, Ottenwälder B, Obermaier B, Tampe J, Heubner D, Wambutt R, Korn B, Klein M, Poustka A. Toward a catalog of human genes and proteins: sequencing and analysis of 500 novel complete protein coding human cDNAs. Genome Res. 2001; 11 (3):422-35.
[14] Clark H F, Gurney A L, Abaya E, Baker K, Baldwin D, Brush J, Chen J, Chow B, Chui C, Crowley C, Currell B, Deuel B, Dowd P, Eaton D, Foster J, Grimaldi C, Gu Q, Hass P E, Heldens S, Huang A, Kim H S, Klimowski L, Jin Y, Johnson S, Lee J, Lewis L, Liao D, Mark M, Robbie E, Sanchez C, Schoenfeld J, Seshagiri S, Simmons L, Singh J, Smith V, Stinson J, Vagts A, Vandlen R, Watanabe C, Wieand D, Woods K, Xie M H, Yansura D, Yi S, Yu G, Yuan J, Zhang M, Zhang Z, Goddard A, Wood W I, Godowski P, Gray A. The secreted protein discovery initiative (SPDI), a large-scale effort to identify novel human secreted and transmembrane proteins: a bioinformatics assessment. Genome Res. 2003; 13 (10):2265-70.
[15] Uhl G R, Liu Q R, Drgon T, Johnson C, Walther D, Rose J E, David S P, Niaura R, Lerman C. Molecular genetics of successful smoking cessation: convergent genome-wide association study results. Arch Gen Psychiatry. 2008; 65 (6):683-93.
[16] Shen S P, Liu W T, Lin Y, Li Y T, Chang C H, Chang F W, Wang L M, Teng S W, Hsuan Y. EphA2 is a biomarker of hMSCs derived from human placenta and umbilical cord. Taiwan J Obstet Gynecol. 2015; 54 (6):749-56.

What is claimed is:

1. A method for determining the risk of preterm labor in a pregnant female subject, comprising detecting the expression level of one or more markers selected from the group consisting of TMEM200A, TMEM108 and GXYLT2, in a sample derived from the pregnant female subject.

2. The method of claim 1, wherein the sample is selected from the group consisting of blood, urine, amniotic fluid, and mesenchymal stem or stromal cells (MSCs) derived from a placenta-related tissue.

3. The method of claim 2, wherein the placenta-related tissue is selected from the group consisting of amniotic membrane, chorionic disk, chorionic membrane, and umbilical cord.

4. The method of claim 1, wherein the expression level is a protein expression level or an mRNA expression level.

5. The method of claim 1, wherein the detecting step is performed through a polymerase chain reaction, a real-time polymerase chain reaction, or a quantitative polymerase chain reaction.

6. The method of claim 1, further comprising comparing the expression level of the marker with a normal expression level.

7. The method of claim 6, wherein the normal expression level is an average expression level of the marker in a plurality of corresponding samples derived from a plurality of pregnant female subjects who deliver at full term.

8. The method of claim 6, wherein a lower expression level of TMEM200A indicates the pregnant female subject is at risk of preterm delivery.

9. The method of claim 8, wherein the lower expression level is an expression level at least 5%, 10%, 20% or 30% lower than the normal expression level.

10. The method of claim 6, wherein a lower expression level of TMEM108 indicates the pregnant female subject is at risk of preterm delivery.

11. The method of claim 10, wherein the lower expression level is an expression level at least 5%, 10%, 20% or 30% lower than the normal expression level.

12. The method of claim 6, wherein a lower expression level of GXYLT2 indicates the pregnant female subject is at risk of preterm delivery.

13. The method of claim 10, wherein the lower expression level is an expression level at least 5%, 10%, 20% or 30% lower than the normal expression level.
